(19) United States
(12) Patent Application Publication
(10) Pub. No.: US 2010/0311638 A1
(43) Pub. Date: Dec. 9, 2010

(54) Method for Altering the Shape of Polymer Particles

(76) Inventor: Paul Tiege, Edmonton (CA)

Correspondence Address: WILLIAM J BUNDREN;THE LAW OFFICE OF WILLIAM J BUNDREN, 734 LaRue Road, Millersville, MD, 21108, US

(21) Appl. No.: 12/447,298

(22) PCT Filed: Oct. 27, 2006
(86) PCT No.: PCT/CA2007/001949

§ 371 Date: Aug. 13, 2010

Related U.S. Application Data
(60) Provisional application No. 60/863,327, filed on Oct. 27, 2006.

(51) Int. Cl. A61K 38/02; C08F 6/00; A61K 31/70; A61K 31/7088; C08L 89/00; C08K 5/01; C08L 29/04

(52) U.S. Cl. 514/1.1; 528/502 R; 514/23; 514/44 R; 528/486; 528/487; 528/490; 524/17; 524/81; 525/56

(57) ABSTRACT

A process for altering the shape of a polymer particle is disclosed. The process comprises suspending polymer particles in a suspending medium at a temperature that effects melting of the polymer and agitating the suspension for a time sufficient to change the shape of the particles. The shape altered particles are combined with a pharmaceutically acceptable agent to give a drug delivery particle system.

### CROSS-REFERENCE TO RELATED APPLICATIONS

This application claims priority under 35 U.S.C. §119(e) to U.S. Provisional Application Ser. No. 60/863,327, filed on Oct. 27, 2006, which is incorporated by reference in its entirety herein.

### TECHNICAL FIELD

The present disclosure is directed to a method for altering the shape of polymer particles by agitating the particles in a suspending solution at an elevated temperature (i.e. at or above the melting point of the polymer) for a period of time necessary to effect a change in shape of the polymer particles.

### BACKGROUND

The shape of a polymer particle may alter the biological, physical, or mechanical properties of the particle. Irregularly shaped particles may have non-predictable surface area-to-particle ratios, hampering the predictability of surface modifications, altering degradation rates, and defeating attempts at sorting for size or other properties. Irregular shapes can have other undesirable physical properties, e.g., they can cause catheter blockage when embolizing a tumor. Removing angles or irregularities from a polymer particle obviates many of these problems.

“Spheronization” is a term commonly understood to mean or to describe a process by which a particle (initially non-spherical in shape) is made spherical. The most common example of this process is the conversion of wet-mass extrudate of microcrystalline cellulose into spheres. Wet-mass extrusion typically involves mixing, at a minimum, microcrystalline cellulose and water to form a paste, then pushing the mixture through an opening to create an extrudate of a desired form. This extrudate can take the form of a strand, and the strand can be cut into particles. These particles are malleable at room temperature and can easily be made spherical by subjecting them, in batch fashion, to the action of a spheronizer. A spheronizer is a metal drum with a rapidly spinning disk on the bottom that converts wet-mass extruded particles into spheres by causing them to hit each other, the wall of the spheronizer, and the spinning disk, deforming the particles randomly until a spherical shape is obtained. This process typically takes less than 15 minutes at room temperature. The rapid spheronization of wet-mass extruded particles is facilitated by the particles' malleability.

### SUMMARY

Altering the shape of a polymer particle is accomplished by immersing one or more initial polymer particles in a suspending medium at a temperature at or above the melting point of the polymer. The particles are agitated at this temperature for a period of time to allow a shape change to occur. In one example, the process can produce a spherical particle (i.e. “spheronization”).

The shape altered polymer particles made by the method of the disclosure have a wide a variety of uses. They can be used, for example, in medical devices, as drug delivery vehicles (particles), as particles for use in chromatography (size exclusion, ion exchange, etc.), and in the manufacturing of various types of plastics.

Provided herein is a method of altering the shape of polymer particles, the method includes suspending the polymer particles in a suspending medium at a temperature that effects melting of the polymer; and agitating the suspension for a time sufficient to effect a change in shape of the particles to result in shape altered polymer particles. In certain embodiments, the method can also include cooling the suspension below the melting temperature of the polymer to maintain the altered shape of the particles. The shape altered particles can further be collected.

The polymer particles can be selected from one or more of polyvinyl alcohol (PVA); polystyrene; polycarbonate; polylactide; polyglycolide; lactide-glycolide copolymers; polycaprolactone; lactide-caprolactone copolymers; polyhydroxybutyrate; polyalkylcyanoacrylates; polyanhydrides; polyorthoesters; albumin; collagen; gelatin; polysaccharides; dextrans; starches; methyl methacrylate; methacrylic acid; hydroxylalkyl acrylates; hydroxylalkyl methacrylates; methylene glycol dimethacrylate; acrylamide; bisacrylamide; cellulose-based polymers; ethylene glycol polymers and copolymers; oxyethylene and oxypropylene polymers; polyvinyl acetate; polyvinylpyrrolidone; polyvinylpyridine; polyanhidrides; and latex.

In certain embodiments, the polymer particle can include a non-biological polymer. In some embodiments, the polymer particles are selected from polylactide, polyglycolide, and poly(lactic-co-glycolic acid) (PLGA). In one embodiment, the polymer particles are PLGA. PLGA can be composed of a ratio of polylactide to polyglycolide from about 90:10, about 75:25. about 65:35, and about 50:50.

The initial polymer particles can be produced by one or more of heat extrusion/pelleting, grinding, and cryo-grinding.

In other embodiments, the polymer particle further comprises a pharmaceutically acceptable agent. This pharmaceutically acceptable agent can be selected from a small molecule (e.g., small molecule drug or prodrug), a carbohydrate, a lipid, a protein, or a nucleic acid.

In the method, the initial particles are suspended in a suspending medium. The suspending medium can be an aqueous medium (e.g., water or saline). In some embodiments, the aqueous medium can further include Tris, Tyrodes, phosphate, citrate, or carbonate buffers. In addition, the aqueous medium can include an additional component that prevents the polymer particles from coalescing or aggregating (e.g., polyvinyl alcohol, a polypeptide, a detergent, or a hydrocarbon).

The shape altered polymer particle can be rigid or elastic and may further be biodegradable. The shape altered polymer particles can be spherical, elliptical, elongated, bowling pin, egg, or oval shaped. In a preferred embodiment the shape altered polymer particles are spherical. The longest dimension of the shape altered polymer particles is about 5 μm to about 5,000 μm. In the case of a spherical particle, the diameter is about 5 μm to about 5,000 μm.

Further provided is a method of preparing a spherical polymer particle, the method includes suspending non-spherical polymer particles in a suspending medium at a temperature that effects melting of the polymer; and agitating the suspension for a time sufficient to effect a change in shape of the particles to result in spherical polymer particles. In certain embodiments, the method can also include cooling the suspension below the melting temperature of the polymer to maintain the spherical shape of the particles. The spherical particles can further be collected.

A drug delivery particle can be prepared through the method of suspending an initial polymer particle in a suspending medium at a temperature that effects melting of the polymer; agitating the suspension for a time sufficient to effect a change in shape of the initial particles to result in shape altered particles; and incorporating a pharmaceutically acceptable agent into the initial or the shape altered particles. The incorporation of a pharmaceutically acceptable agent into the polymer particles can occur during production of the initial polymer particles, during agitation of the suspension, and/or after obtaining shape altered particles. In certain embodiments, the drug delivery particle is spherical.

A spherical particle can be prepared through the method of suspending a polymer particle in a suspending medium at a temperature that effects melting of the polymer; and agitating the suspension for a time sufficient to effect a change in shape of the particles to result in spherical particles.

The details of one or more embodiments of the invention are set forth in the description below. Other features, objects, and advantages of the invention will be apparent from the description and from the claims.

### DETAILED DESCRIPTION OF THE DRAWINGS

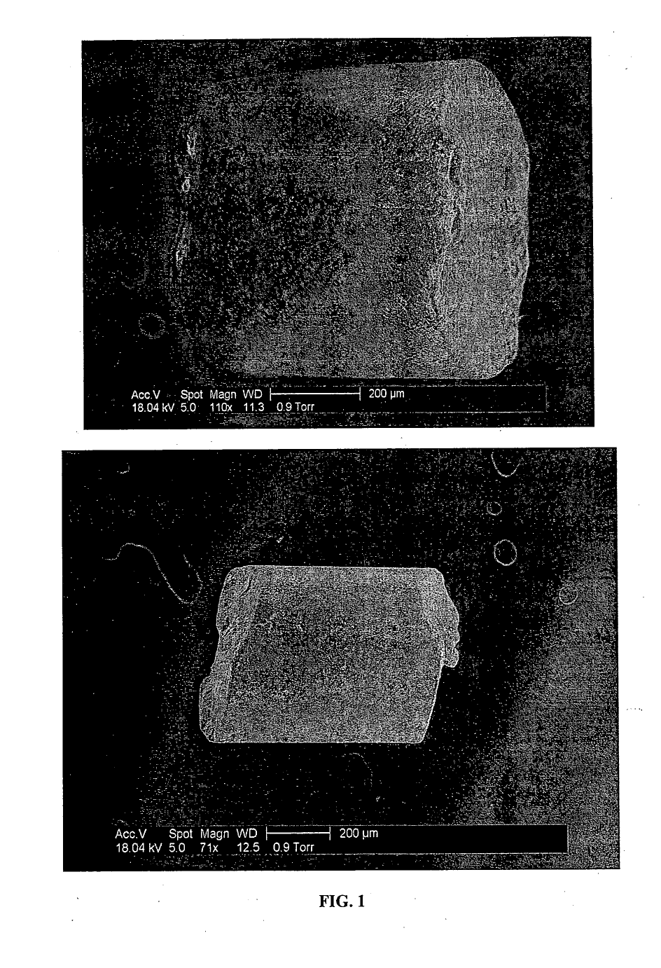

FIG. 1. is an SEM image of an example initial polymer particle. In this example, the initial polymer particle is a cylinder of 75:25 PLGA.

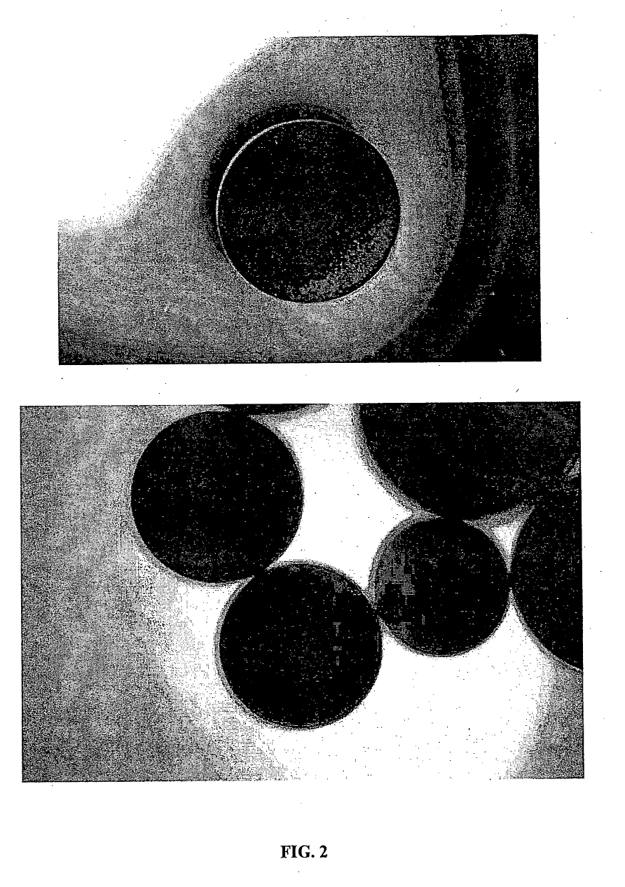

FIG. 2 is an photographic image of a shape altered polymer particle. In this example, the shape altered polymer particle is a spherical particle of 75:25 PLGA. The photographs were taken on a Hund H500 light microscope with a Nikon D70 digital SLR camera, with a microscope adapter. The photographs are taken at 40× magnification.

### DETAILED DESCRIPTION

As used herein, “agitation” or “agitating” includes stirring, shaking, end-over-end mixing, aspiration, and/or bubbling with gas (e.g., air, nitrogen, or an inert gas such as helium or argon).

As used herein, “suspending medium” refers to an aqueous of non-aqueous solution in which the polymer particles are not soluble.

Provided herein are methods of altering the shape of polymer particles, for example, from non-spherical to spherical. The process of altering the shape of a polymer particle occurs because the polymer particle is heated at or above its melting point in a solution in which the polymer is not soluble. This allows the polymer to act as a fluid droplet. A fluid moving independently in another fluid is subject to surface tension, a force that acts with even distribution across the surface, and the pressure of the second fluid. A molten droplet of polymer, under the appropriate conditions, will assume a lowest-energy shape that is the result of the interaction of the surface tension of the polymer and the pressure of the aqueous solution that surrounds it. The pressure of the aqueous solution acts with equal force at all points on the surface of the droplet, and the surface tension of the droplet will act to create the lowest-energy conformation. One example of this phenomenon is the shape of raindrops. Raindrops are different shapes depending on their size; small raindrops (radius <1 millimeter (mm)) are spherical, while larger ones assume a shape more like that of a hamburger bun. The shape results from a tug-of-war between two forces: the surface tension of the water and the pressure of the air pushing up against the bottom of the drop as it falls. When the drop is small, surface tension wins and pulls the drop into a spherical shape. With increasing size, the fall velocity increases and the pressure on the bottom increases causing the raindrop to flatten and even develop a depression.

The method of altering the shape of a polymer particle is not limited by the material that may be used in forming the initial particles. Any material may be used provided that it is thermally plastic and able to undergo shape change when heated and to maintain a final shape when cooled. The materials most amenable to use are biological and non-biological polymers. Of particular interest are non-biological polymers. These polymers may include chemically functionalized polymers, chemically inert polymers, biologically active polymers, biodegradable polymers, and labeled polymers (including those polymers labeled with a drug, dye, isotope, chelate, antibody, etc.). Polymers can also be copolymers, block copolymers, etc.

Exemplary non-biological polymers include polyvinyl alcohol (PVA); polystyrene; polycarbonate; polylactide; polyglycolide; lactide-glycolide copolymers; polycaprolactone; lactide-caprolactone copolymers; lactide-glycolide caprolactone copolymers; polyhydroxybutyrate; polyalkylcyanoacrylates; polyanhydrides; polyorthoesters; methyl methacrylate; methacrylic acid; hydroxylalkyl acrylates; hydroxylalkyl methacrylates; methylene glycol dimethacrylate; acrylamide; bisacrylamide; ethylene glycol polymers and copolymers; oxyethylene and oxypropylene polymers; polyvinyl acetate; polyvinylpyrrolidone; polyvinylpyridine; polyanhidrides (e.g., maleic anhydride); and latex.

Non-limiting examples of biological polymers include albumin; collagen; gelatin; polysaccharides; dextrans; starches; and cellulose-based polymers.

In certain embodiments the polymer is polylactide. In another embodiment the polymer is polyglycolide. In some embodiments the polymer is poly(lactic-co-glycolic acid) (PLGA). The ratio of polylactide to polyglycolide in PLGA can be about 90:10, about 75:25, about 65:35, or about 50:50.

The initial polymer particles can be initially produced by various methods including heat extrusion through a dye followed by cutting to form particles. The particles can also be produced using a grinding method that breaks the polymer into chunks. In one embodiment the polymer particles are produced by heat extrusion/pelleting. In another embodiment the polymer particles are produced by grinding. In further embodiments the polymer particles are produced by cryo-grinding.

Initially, the polymer particles can be of any shape, e.g., without limitation, irregular and without any shape, barrel-shaped, cylindrical, cubic, rhomboid or amorphous. The particles can be of any convenient size and are generally in the range of about 5 Tm to about 5,000 Tm in their longest axis (dimension) (e.g., about 5 Tm to about 2,500 Tm, about 5 Tm to about 1000 Tm, about 5 Tm to about 500 Tm, about 5 Tm to about 100 Tm, about 25 Tm to about 5,000 Tm, about 100 Tm to about 5,000 Tm, about 500 Tm to about 5,000 Tm, about 1,000 Tm to about 5,000 Tm, about 2,500 Tm to about 5,000 Tm, about 50 Tm to about 2,500 Tm, about 100 Tm to about 1,500 Tm, and about 250 Tm to about 750 Tm). Particles of varying size and shape can be altered at the same time. However, it is preferred to alter the shape of particles of similar shape and size in any given procedure. Thus, in certain embodiments the polymer particles are of a similar size.

Initial polymer particles are suspended in a suspending medium. The suspending medium can be an aqueous or non-aqueous fluid medium. In certain embodiments, the suspending medium is an aqueous solution. Aqueous solutions include any aqueous solution in which the described alteration can occur. Such solutions include, for example, water or saline, and can contain buffers such as Tris, Tyrodes, phosphate, citrate, or carbonate. The solutions can contain additional additives that prevent the particles from coalescing or aggregating; for example, polyvinyl alcohol (PVA), one or more polypeptides (e.g., bovine serum albumin), detergents, and hydrocarbons. In certain embodiments the additive is polyvinyl alcohol.

Altering the shape of an initial polymer particle can be accomplished by immersing the initial polymer particles in the suspending medium at a temperature at or above the melting point of the polymer. One of ordinary skill in the art will understand that the necessary temperature required to achieve an alteration in the shape of a polymer particle will vary based on the polymer used and the final shape desired. For example, spherinization of non-spherical polymer particles of 75:25 PLGA can occur at temperatures from about 70° C. to about 90° C. In another example, non-spherical particles of 50:50 PLGA can be made spherical at temperatures from about 60° C. to about 70° C.

The particles are agitated at this temperature for a period of time to allow a shape change to occur. In one example, spherical particles are produced by stirring a suspension of polymer particles with a magnetic stirbar. In another example, ovoid particles are produced by stirring a suspension of polymer particles with a magnetic stirbar, at a higher stir rate than that used to produce spherical particles. In another example, spherical particles are produced by end-over-end mixing in a closed vial. In some cases, the polymer particles can be agitated from one to 24 hours (e.g., 4 to 24 hours, 4 to 18 hours, 4 to 12 hours, 4 to 8 hours, 6 to 20 hours, 8 to 24 hours, 10 to 16 hours, 2 to 3 hours, 2 to 6 hours, 5 to 15 hours, and 8 to 12 hours). For example, spheronization of 75:25 PLGA cylinders of approximately 1 mm can be spherionized in about 4 to 18.5 hours at 80° C.

Altered particles can have many shapes, including spherical, elliptical, elongated, bowling pin, egg, and oval. Typically, the final shape is continuously smooth with no sharp edges. Thus, the method can also be used to smooth a particle to remove one or more edges.

In certain embodiments, the altered particles, are spherical. Spherical products of the method can be of any useful diameter but are generally in the range of about 5 Tm to about 5,000 Tm (e.g., about 5 Tm to about 2,500 Tm, about 5 Tm to about 1000 Tm, about 5 Tm to about 500 Tm, about 5 Tm to about 100 Tm, about 25 Tm to about 5,000 Tm, about 100 Tm to about 5,000 Tm, about 500 Tm to about 5,000 Tm, about 1,000 Tm to about 5,000 Tm, about 2,500 Tm to about 5,000 Tm, about 50 Tm to about 2,500 Tm, about 100 Tm to about 1,500 Tm, and about 250 Tm to about 750 Tm). Non-spherical products of the method are generally about 5 Tm to about 5,000 Tm in their longest dimension (e.g., about 5 Tm to about 2,500 Tm, about 5 Tm to about 1000 Tm, about 5 Tm to about 500 Tm, about 5 Tm to about 100 Tm, about 25 Tm to about 5,000 Tm, about 100 Tm to about 5,000 Tm, about 500 Tm to about 5,000 Tm, about 1,000 Tm to about 5,000 Tm, about 2,500 Tm to about 5,000 Tm, about 50 Tm to about 2,500 Tm, about 100 Tm to about 1,500 Tm, and about 250 Tm to about 750 Tm). The final shape of a polymer particle can be determined, for example, visually with the aid of a light microscope, using laser diffraction, by digital photographic shape analysis software, or by other methods known in the art.

In one preferred embodiment, the process can produce a spherical particle (i.e. “spheronization”). The spheronization process converts initial polymer particles created by various processes including melt-extrusion/pelleting to discrete, spherical particles. Melt-extrusion (or heat-extrusion)/pelleting is the process in which a polymer is melted and the melted polymer is pushed through a die to form an extrudate. A polymer, e.g., poly(lactic-co-glycolic acid) (PGLA), is introduced into a screw and heated as it is compacted and pushed by the screw. The molten polymer is pushed through a hole located at the end of the screw, creating a strand. This strand is then cooled and introduced into a pelleter, which consists of a spinning cutting head or an oscillating “guillotine” cutting blade that cuts the strand into pellets. The initial polymer pellets (i.e. particles) are converted to a spherical shape by introducing them into an aqueous or non-aqueous fluid medium, depending on the composition of the polymer, at a defined temperature and agitated for a given period of time. The temperature chosen should be at or above the melting temperature of the polymer. Once a spherical shape has been achieved, the suspension is cooled below the melting temperature of the polymer to maintain the spherical shape of the particles. For example, the shape altered polymer particles can be cooled slowly at room temperature with stirring, quickly cooled using, for example, an ice bath of liquid nitrogen, or by other means known in the art.

The shape altered polymer particles are then collected and stored below the melting temperature of the polymer. Polymer particles may be collected by any means known in the art, e.g., filtration.

The altered polymer particles made by the method of the disclosure have a wide a variety of uses. They can be used, for example, in medical devices, as drug delivery vehicles (particles), as particles for use in chromatography (size exclusion, ion exchange, etc.), and in the manufacturing of various types of plastics. Spherical polymer particles are well-known in the art as medical devices (Embosphere Microspheres (Biosphere Medical)), Contour SE (Boston Scientific), and as drug delivery vehicles (Lupron Depot (TAP Pharma)). PLGA has also been used as a drug delivery vehicle, most commonly in depot applications, including, for example: Risperdal Consta (atypical psychosis; Janssen) in which 25 mg, 37.5 mg or 50 mg of risperidone are encapsulated in PLGA microspheres at a concentration of 381 mg of risperidone per gram of PLGA; Lupron Depot, 3-month, 11.25 mg (NDA 20-708) a drug product comprised of inter alfa 11.25 mg leuprolide acetate and a carrier of 99 mg poly(lactic acid) that is administered as an intramuscular injection every three months for the management of endometriosis and anemia caused by uterine fibroids; Sandostatin LAR (somatostatin mimic; Novartis) in which 11 mg, 22 mg or 34 mg of octreotide acetate are encapsulated in 189 mg, 378 mg or 566 mg of PLGA respectively; and Trelstar Depot (LHRH agonist, Watson Labs) in which 3.75 mg of triptorelin pamoate are encapsulated in 170 mg of PLGA. In addition, the polymer particles can be used to deliver a chemotherapeutic agent to a tumor via a catheter (see, e.g., U.S. Pat. Nos. 6,900,352 and 6,887,474 and U.S. Publication Nos. 2007/0098724, 2005/0287189, 2005/0287145, 2005/0079179, 2003/0082224, and 2002/0168366).

When the polymer particles are used as a medical device or as a drug delivery agent, the polymer particles can further include one or more additional components, e.g., a pharmaceutically acceptable agent (e.g., small molecule drug or prodrug, chemotherapeutic, HSA, collagen, diagnostic or imaging agent, carbohydrate, lipid, polypeptide, or nucleic acid (e.g., SiRNA). One suitable example of a polypeptide is an antibody or antibody fragment. The additional component may be incorporated either physically (e.g., encapsulated, absorbed, or coated on the surface) or chemically (e.g., covalently or non-covalently bound) into the polymer particle at any point in the production of the polymer particle. For example, the additional agent may be incorporated during synthesis of the polymer, during production of the initial polymer particle, during the process of altering the shape of the particle, or after the final shape of the polymer particle has been achieved. In some instances, the additional agent is covalently or non-covalently bound to the polymer particle. In other instances the additional agent is encapsulated in the polymer particle. Suitable linkers for chemically linking the additional component are well known and can be biodegradable.

### EXAMPLES

### Example 1

In this representative example, the ratio of suspending fluid to mass of particles was 100 mL:1 g. Spheronization was accomplished in 4 hours. Particles to be spheronized were made of PLGA composed of 75 mol % lactic acid residues and 25 mol % glycolic acid residues (75:25 lactide/glycolide).

Materials
75:25 PLGA pellets: pellets made by melt-extrusion/pelleting process, approximately 1 mm in length. Polymer was Boehringer Ingelheim Resomer RG 755S (75:25 lactide/glycolide; IV ˜0.7)
Programmable stirrer/hotplate
Spinner flask
PVA, Aldrich 87-89% hydrolysed 31 000-50 000 MW Lot # 00514CQ

Method
1. To a 125-mL spinner flask charged with 100 mL of 0.2% PVA solution (pH 6-7) stirring at 20-25° C. at 130 rpm was added ˜1 g of 75:25 PLGA pellets.
2. Heating was initiated and the solution was heated to 80° C. over 15-30 minutes. Stirring was maintained.
3. The temperature was maintained at 80° C. for 4 hours. Stirring was maintained.
4. Heating was discontinued and the solution temperature was allowed to decrease to 20-25° C. Stirring was maintained.
5. Microspheres were collected by filtration and washed copiously with deionized water to remove PVA.
6. Microspheres were dried and stored at 4° C.

### Example 2

In this representative example, the ratio of suspending fluid to mass of particles was 500 mL:152 g (3.3 mL:1 g). Spheronization was accomplished in 4 hours. Particles to be spheronized were made of PLGA composed of 75 mol % lactic acid residues and 25 mol % glycolic acid residues (75:25 lactide/glycolide).
1. PLGA cylinders from RG 755S (Resomer™, Boehringer Ingelheim) were transferred to a 500-mL spinner flask containing 500 mL 0.2% PVA at room temperature and stirred at 280 rpm.
2. The temperature was increased to 80° C. and maintained at 80° C. for four hours with constant stirring.
3. Heating was discontinued and the solution temperature was allowed to decrease to 20-25° C. Stirring was maintained.
4. Microspheres were collected by filtration and washed copiously with deionized water to remove PVA.

### Example 3

In this representative example, the ratio of suspending fluid to mass of particles is 6 mL:0.6 g (10 mL:1 g). Spheronization was accomplished by end-over-end mixing. Particles to be spheronized were made of PLGA composed of 50 mol % lactic acid residues and 50 mol % glycolic acid residues (50:50 lactide/glycolide).
1. 600 mg of particles from melt-extruded 50:50 PLGA (RG 504H; Resomer™, Boehringer Ingelheim) were added to a 10-mL vial containing 6 mL of 0.2% PVA at room temperature and the vial sealed.
2. The vial was attached to an end-over-end mixer, the mixer placed inside an oven maintained at 65° C. and the mixer was powered on.
3. End-over-end agitation was maintained for 4 hours, after which the particles looked perfectly round by eye.

### Example 4

In this representative example, the ratio of suspending fluid to mass of particles is 100 mL:0.5 g (200 mL:1 g). Change in shape from amorphous to ovoid was accomplished in 18.5 hours. Particles undergoing shape change were made of PLGA composed of 75 mol % lactic acid residues and 25 mol % glycolic acid residues (75:25 lactide/glycolide).
1. To a 250-mL beaker containing 100 mL 0.2% PVA solution at room temperature and stirring at 270 rpm was added 501 mg of PLGA pellets.
2. The temperature of the stirred suspension was raised from 21° C. to 70° C. and the conditions maintained for approximately 18.5 hours.
3. After 18.5 hours, the pellets had uniformly become ovoid, like “Smarties™.”

A number of embodiments of the invention have been described. Nevertheless, it will be understood that various modifications may be made without departing from the spirit and scope of the invention. Accordingly, other embodiments are within the scope of the following claims.

What is claimed is:

1. A method of altering the shape of polymer particles, the method comprising:
a) suspending the polymer particles in a suspending medium at a temperature that effects melting of the polymer; and
b) agitating the suspension for a time sufficient to effect a change, in shape of the particles to result in shape altered polymer particles.

2. The method of claim 1, wherein the polymer particle comprises a non-biological polymer.

3. The method of claim 1, wherein the polymer particle further comprises a pharmaceutically acceptable agent.

4. The method of claim 3, wherein the pharmaceutically acceptable agent is selected from a small molecule, a carbohydrate, a lipid, a protein, or a nucleic acid.

5. The method of claim 1, wherein the shape altered polymer particle is rigid.

6. The method of claim 1, wherein the shape altered polymer particle is elastic.

7. The method of claim 1, wherein the shape altered polymer particle is biodegradable.

8. The method of claim 1, wherein the polymer particles are one or more of polyvinyl alcohol (PVA); polystyrene; polycarbonate; polylactide; polyglycolide; lactide-glycolide copolymers; polycaprolactone; lactide-caprolactone copolymers; polyhydroxybutyrate; polyalkylcyanoacrylates; polyanhydrides; polyorthoesters; albumin; collagen; gelatin; polysaccharides; dextrans; starches; methyl methacrylate; methacrylic acid; hydroxylalkyl acrylates; hydroxylalkyl methacrylates; methylene glycol dimethacrylate; acrylamide; bisacrylamide; cellulose-based polymers; ethylene glycol polymers and copolymers; oxyethylene and oxypropylene polymers; polyvinyl acetate; polyvinylpyrrolidone; polyvinylpyridine; polyanhidrides; and latex.

9. The method of claim 8, wherein the polymer particles are selected from polylactide, polyglycolide, and poly(lactic-co-glycolic acid) (PLGA).

10. The method of claim 9, wherein the polymer particles are PLGA.

11. The method of claim 10, wherein the ratio of polylactide to polyglycolide in the PLGA is selected from about 90:10, about 75:25. about 65:35, and about 50:50.

12. The method of claim 1, wherein the polymer particles are produced by one or more of heat extrusion/pelleting, grinding, and cryo-grinding.

13. The method of claim 1, wherein the suspending medium is an aqueous medium.

14. The method of claim 13, wherein the aqueous medium is selected from water or saline.

15. The method of claim 13, wherein the aqueous medium further comprises one or more of Tris, Tyrodes, phosphate, citrate, or carbonate buffers.

16. The method of claim 13, wherein the aqueous medium further comprises an additional additive, wherein the additive prevents the polymer particles from coalescing or aggregating.

17. The method of claim 16, wherein the additional additive is selected from one or more of a polyvinyl alcohol, a polypeptide, a detergent, or a hydrocarbon.

18. The method of claim 1, wherein the method further comprises:
c) cooling the suspension below the melting temperature of the polymer to maintain the altered shape of the particles.

19. The method of claim 1, wherein the method further comprises collecting the shape altered particles.

20. The method of claim 1, wherein the altered shape of the polymer particles is spherical, elliptical, elongated, bowling pin, egg, or oval.

21. The method of claim 20, wherein the longest dimension of the altered particles is about 5 μm to about 5,000 μm.

22. The method of claim 20, wherein the altered shape of the particles is spherical.

23. The method of claim 22, wherein the spherical polymer particles have a diameter from about 5 μm to about 5,000 μm.

24. A method of preparing spherical polymer particles, the method comprising:
a) suspending non-spherical polymer particles in a suspending medium at a temperature that effects melting of the polymer; and
b) agitating the suspension for a time sufficient to effect a change in shape of the particles to result in spherical polymer particles.

25. The method of claim 24, wherein the method further comprises:
c) cooling the suspension below the melting temperature of the polymer to maintain the spherical shape of the particles.

26. The method, of claim 24, wherein the method further comprises collecting the spherical particles.

27. A method of making a drug delivery particle, the method comprising:
a) suspending an initial polymer particle in a suspending medium at a temperature that effects melting of the polymer;
b) agitating the suspension for a time sufficient to effect a change in shape of the initial particles to result in shape altered particles; and
c) incorporating a pharmaceutically acceptable agent into the initial or the shape altered particles.

28. The method of claim 27, wherein the incorporation of a pharmaceutically acceptable agent is during production of the initial polymer particles.

29. The method of claim 27, wherein the incorporation of a pharmaceutically acceptable agent is during step b).

30. The method of claim 27, wherein the incorporation of a pharmaceutically acceptable agent occurs after obtaining shape altered particles.

31. A spherical particle produced by the process of:
a) suspending a polymer particle in a suspending medium at a temperature that effects melting of the polymer; and
b) agitating the suspension for a time sufficient to effect a change in shape of the particles to result in spherical particles

32. A spherical drug delivery particle produced by the process of:
a) suspending an initial polymer particle in a suspending medium at a temperature that effects melting of the polymer;
b) agitating the suspension for a time sufficient to effect a change in shape of the initial particles; and
c) incorporating a pharmaceutically acceptable agent into the initial or spherical particles.
